(19) United States
(12) Patent Application Publication
(10) Pub. No.: US 2015/0112181 A1
(43) Pub. Date: Apr. 23, 2015

(54) WIDEBAND ULTRASONIC PROBE FOR PHOTOACOUSTIC IMAGE AND ULTRASOUND IMAGE

(71) Applicant: Samsung Electronics Co., Ltd., Suwon-si (KR)

(72) Inventors: Yong-seop YOON, Seoul (KR); Sung-chan KANG, Hwaseong-si (KR); Jong-seok KIM, Hwaseong-si (KR); Sang-ha PARK, Seoul (KR); Seok-whan CHUNG, Hwaseong-si (KR)

(73) Assignee: Samsung Electronics Co., Ltd., Suwon-si (KR)

(21) Appl. No.: 14/249,509

(22) Filed: Apr. 10, 2014

(30) Foreign Application Priority Data
Oct. 22, 2013 (KR) 10-2013-0126103

(51) Int. Cl. A61B 5/00

(52) U.S. Cl. CPC A61B 5/0095
USPC 600/407

(57) ABSTRACT

Provided are a wideband ultrasonic probe for a photoacoustic image and an ultrasound image. The wideband ultrasonic probe includes a first ultrasonic transducer array and a second ultrasonic transducer array that are disposed on a substrate; and a laser apparatus that comprises a laser irradiator configured to irradiate a laser light onto a diagnosis object, wherein the first ultrasonic transducer array receives a first ultrasonic wave which is generated from the diagnosis object on which the laser light is irradiated, and the second ultrasonic transducer array transmits a high frequency bandwidth ultrasonic wave toward the diagnosis object and receives a second ultrasonic wave that is reflected by the diagnosis object.

### CROSS-REFERENCE TO RELATED APPLICATIONS

This application claims priority from Korean Patent Application No. 10-2013-0126103, filed on Oct. 22, 2013, in the Korean Intellectual Property Office, the disclosure of which is incorporated herein by reference in its entirety.

### BACKGROUND

1. Field

Exemplary embodiments relate to ultrasonic probes that facilitate a correct diagnosis by realizing a functional image on a morphological image by combining an ultrasound image with a photoacoustic image.

2. Description of the Related Art

An ultrasonic probe is used for analyzing morphological characteristics of an organ and a texture of a human body by realizing an image by receiving echo signals reflected by the human body after transmitting an ultrasonic wave. In the case of diagnosing a shallow texture depth (for example, breast), a high resolution image is realized by using a probe of a high frequency bandwidth (e.g., 5-13 MHz), and in the case of diagnosing a deep texture depth (for example, abdomen), a probe of a low frequency bandwidth (e.g., 2-7 MHz) is used. That is, a probe is selected according to the application of diagnosis, thereby increasing a quality of an image.

However, although the quality of images is improved, the accuracy of diagnosis for an early stage of cancer in order to differentiate between a malignant tissue and a benign tissue is still low due to the limit of a morphological image that is acquired based on an ultrasonic transmission and an ultrasonic reception.

Recently, a technology of applying a photoacoustic technique to a diagnosis has been developed, that is, a functional image is realized by measuring a photo characteristic of a texture by receiving a ultrasonic wave that is generated by irradiating light (laser light) toward the texture of a human body. Studies have been actively performed to increase an accuracy of diagnosis by simultaneously providing a morphological image and a functional image by combining a photoacoustic image and an ultrasound image based on an ultrasonic system.

However, the ultrasonic frequency bandwidth that is used in the generation of an ultrasound image and the frequency bandwidth that is generated when the photoacoustic image is generated may be different from each other. For example, an ultrasonic probe that is used for generating an ultrasound image for a breast cancer diagnosis uses a general high frequency bandwidth, and a probe for generating a photoacoustic image uses a low frequency bandwidth. Thus, in order to simultaneously obtain an ultrasound image and a photoacoustic image, a wideband probe that covers both low and high frequency bandwidths is needed.

Even though a method of combining a low frequency probe and a high frequency probe after respectively manufacturing the low and high frequency probes has been proposed, costs for manufacturing the probes are increased, and there is a difficulty in matching an ultrasound image with a photoacoustic image.

### SUMMARY

Provided are wideband ultrasonic probes that receive low and high frequency bandwidth ultrasonic waves at a same space using a capacitive micromachined ultrasonic transducer (CMUT), and in particular, increase an ultrasonic wave receiving sensitivity when an ultrasound image and a photoacoustic image are generated.

Additional aspects will be set forth in part in the description which follows and, in part, will be apparent from the description, or may be learned by practice of the presented exemplary embodiments.

According to an aspect of one or more exemplary embodiments, an ultrasonic probe includes: a first ultrasonic transducer array and a second ultrasonic transducer array that are disposed on a substrate; and a laser apparatus that comprises a laser irradiator configured to irradiate a laser light onto a diagnosis object, wherein the first ultrasonic transducer array is configured to receive a first ultrasonic wave which is generated from the diagnosis object on which the laser light is irradiated, and the second ultrasonic transducer array is configured to transmit a high frequency bandwidth ultrasonic wave toward the diagnosis object and to receive a second ultrasonic wave that is reflected by the diagnosis object.

The first ultrasonic transducer array may include a plurality of first ultrasonic transducer chips, and the second ultrasonic transducer array may include a plurality of second ultrasonic transducer chips, wherein each of the first and each of the second ultrasonic transducer chips is a capacitive micromachined ultrasonic transducer (CMUT) chip.

Each of first ultrasonic transducer chips may be disposed on a first region of the substrate and each of the second ultrasonic transducer chips may be disposed on a second region of the substrate, and the second region may be adjacent to the first region.

The first and second ultrasonic transducer chips may be alternately disposed.

The first ultrasonic transducer array may be further configured to receive a frequency bandwidth in a range from about 0.5 to about 4 MHz.

The second ultrasonic transducer array may be further configured to receive a frequency bandwidth in a range from about 5 to about 18 MHz.

Cavities between an upper electrode and a lower electrode of the first ultrasonic transducer chip may have a first height that is smaller than a second height of cavities between an upper electrode and a lower electrode of the second ultrasonic transducer chip.

The first height may be in a range from about 10 nm to about 100 nm.

The laser apparatus may include a pulse laser.

The pulse laser may have a pulse width which is in a range of between about 1 picosecond and 1000 nanoseconds.

The ultrasonic probe may further include: a first signal processor configured to generate a first image by receiving, from the first ultrasonic transducer array, an electrical signal which corresponds to the first ultrasonic wave; a second signal processor configured to generate a second image by receiving, from the second ultrasonic transducer array, an electrical signal which corresponds to the second ultrasonic wave; and an image combiner configured to generate a third image by combining the first image with the second image.

The ultrasonic probe may further include a display device configured to display at least one from among the first image, the second image, and the third image.

### BRIEF DESCRIPTION OF THE DRAWINGS

These and/or other aspects will become apparent and more readily appreciated from the following description of exemplary embodiments, taken in conjunction with the accompanying drawings in which:

FIG. 1 is a schematic plan view of a structure of a wideband ultrasonic probe for a photoacoustic image and ultrasound image, according to one or more exemplary embodiments;

FIG. 2 is a cross-sectional view taken along line II-II′ of FIG. 1;

FIG. 3 is a schematic block diagram of a wideband ultrasonic probe for a photoacoustic image and ultrasound image, according to one or more exemplary embodiments; and

FIG. 4 is a schematic plan view of a structure of a wideband ultrasonic probe for a photoacoustic image and ultrasound image, according to one or more exemplary embodiments.

### DETAILED DESCRIPTION

Reference will now be made in detail to exemplary embodiments, examples of which are illustrated in the accompanying drawings. In the drawings, thicknesses may be exaggerated for clarity of layers and regions. The exemplary embodiments are amenable to various modifications and may be embodied in many different forms. When a layer, a film, a region, or a panel is referred to as being “on” another element, it can be directly on the other layer or substrate, or intervening layers may also be present. Like numerals refer to like elements throughout the description of the figures. Like reference numerals are used to indicate elements that are substantially identical to each other, and thus, the detailed description thereof will be omitted.

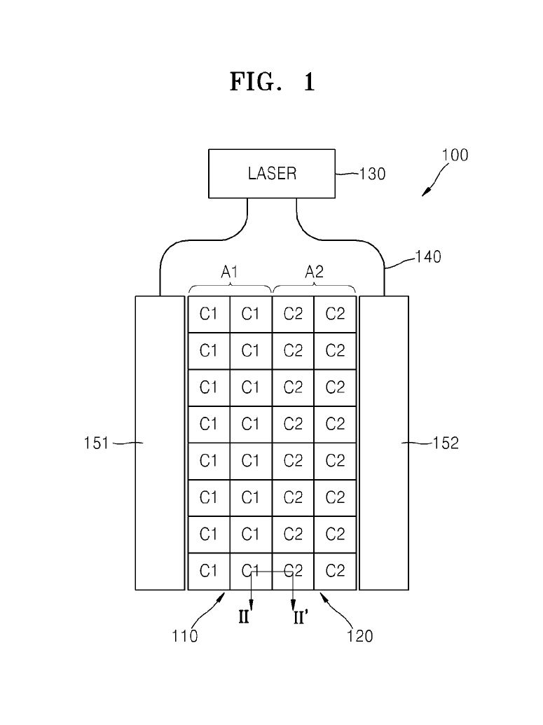

FIG. 1 is a schematic plan view of a structure of a wideband ultrasonic probe 100 for a photoacoustic image and ultrasound image, according to one or more exemplary embodiments. The photoacoustic and ultrasound image is referred to as a combining image of the photoacoustic image and the ultrasound image.

Referring to FIG. 1, the ultrasonic probe 100 may include a first ultrasonic transducer array 110 that is disposed in a first region A1, a second ultrasonic transducer array 120 that is disposed in a second region A2, a first laser irradiation unit (also referred to as a “first laser irradiator” and/or as a “first laser irradiation component”) 151 that is disposed at an outer region of the first ultrasonic transducer array 110, and a second laser irradiation unit (also referred to as a “second laser irradiator” and/or as a “second laser irradiation component”) 152 that is disposed at an outer region of the second ultrasonic transducer array 120. The first region A1 and the second region A2 are adjacent to each other.

The first and second laser irradiation units 151 and 152 are disposed in a probe housing (not shown) together with the first and second ultrasonic transducer arrays 110 and 120.

A laser light which is generated from a single laser generator 130 is divided by optical fibers 140 to the first and second laser irradiation units 151 and 152, and is irradiated onto a diagnosis object. The first and second laser irradiation units 151 and 152 may be optical prisms. The ultrasonic probe 100 may include one of the first and second laser irradiation units 151 and 152. The use of the two laser irradiation units 151 and 152 is to irradiate a uniform laser light onto a target location, and thus, to obtain a uniform intensity of acoustic wave that is generated from a diagnosis object.

The laser generator 130, the optical fibers 140, and the first and second laser irradiation units 151 and 152 constitute a laser apparatus.

The laser generator 130 may be a solid pulse laser, for example, an Nd:YAG pulse laser. A pulse width of a laser light from the laser generator 130 may have a nano size or a pico size, i.e., the pulse width may fall within a range of between about 1 picosecond and 1000 nanoseconds.

The first region A1 and the second region A2 are disposed to be parallel to each other. The first ultrasonic transducer array 110 may include a plurality of first capacitive micro-machined ultrasonic transducer (CMUT) chips C1. Each of the first CMUT chips C1 may include a plurality of elements.

The second ultrasonic transducer array 120 may include a plurality of second CMUT chips C2. Each of the second CMUT chips C2 may include a plurality of elements.

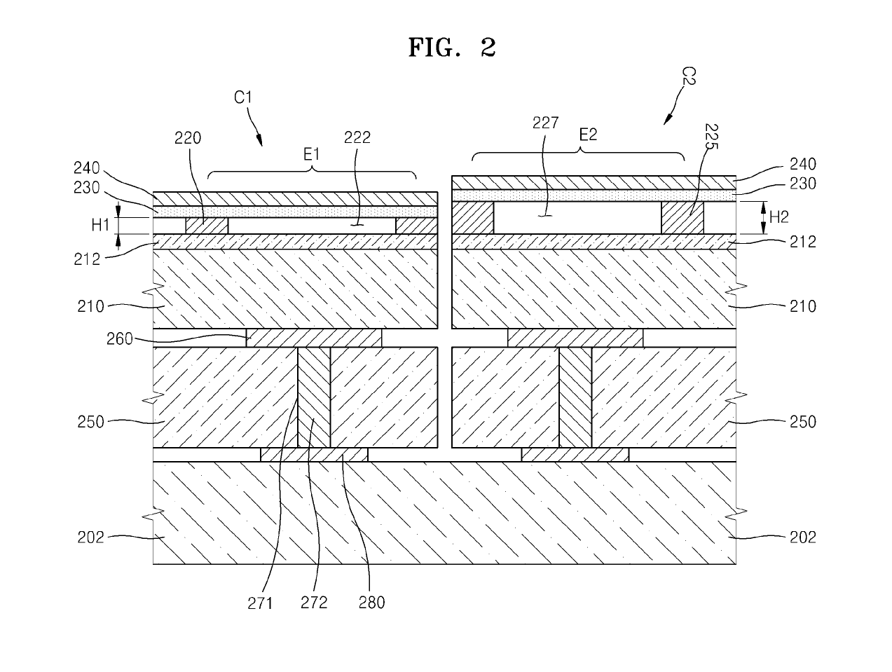

FIG. 2 is a cross-sectional view taken along line II-II′ of FIG. 1.

Referring to FIGS. 1 and 2, a plurality of first CMUT chips C1 and a plurality of second CMUT chips C2 are adjacently disposed on an ASIC substrate 202.

FIG. 2 shows a cross-sectional view of structures of a first element E1 of the first CMUT chip C1 and a second element E2 of the second CMUT chip C2 that are neighboring each other at a boundary between the first region A1 and the second region A2.

The first CMUT chip C1 includes a device substrate 210, an ultrasonic transducer structure on the device substrate 210, and an electrode pad substrate 250 that is provided below the device substrate 210. The ultrasonic transducer structure includes supporters 220, a membrane 230, and an upper electrode 240 that are provided above the device substrate 210. The device substrate 210 may perform as a lower electrode. For example, the device substrate 210 may be a low resistance silicon substrate that is highly doped with a dopant.

An upper insulating layer 212 may be formed on an upper surface of the device substrate 210. The upper insulating layer 212 may be formed of a silicon oxide.

The supporters 220 that define a plurality of cavities 222 are provided on the upper insulating layer 212. The supporters 220 may be formed of a silicon oxide. The membrane 230 that covers the cavities 222 is formed on the supporters 220. The membrane 230 may be formed of silicon, but is not limited thereto. The upper electrode 240 is provided on an upper surface of the membrane 230. The upper electrode 240 may be a metal film formed by sputtering aluminum on the membrane 230.

The electrode pad substrate 250 is provided below the device substrate 210. The electrode pad substrate 250 supplies electricity to the device substrate 210 that performs as a lower electrode. The electrode pad substrate 250 may be, for example, a silicon substrate, but is not limited thereto. The device substrate 210 and the electrode pad substrate 250 are combined by using a bonding layer 260 which is disposed therebetween. The bonding layer 260 is formed to correspond to each of the element regions. The bonding layer 260 may be formed of a material that forms a eutectic bonding between two metals. For example, the bonding layer 260 may be an Au—Sn bonding layer.

A through hole 271 that is formed to correspond to the bonding layer 260 may be formed in the electrode pad substrate 250, and the through hole 271 may be filled with a via metal 272. The via metal 272 is electrically connected to the bonding layer 260. An electrode pad 280 that contacts the via metal 272 is formed under the electrode pad substrate 250. Electricity supplied to the electrode pad 280 is transmitted to the device substrate 210 through the via metal 272 and the bonding layer 260. The upper electrode 240 may be a common electrode. Electricity may be supplied to the upper electrode 240 through an additional via metal (not shown) which is formed in the electrode pad substrate 250 and the device substrate 210, but the description thereof will be omitted.

The electrode pad substrate 250 may be a silicon substrate. If the electrode pad substrate 250 is a conductive silicon substrate, an insulating film (not shown) that insulates the bonding layer 260, the via metal 272, and the electrode pad 280 from the electrode pad substrate 250 may further be formed.

The second CMUT chip C2 has a structure which is almost identical to that of the first CMUT chips C1, and thus, like reference numerals are used to indicate substantially the same elements, and the description thereof will be omitted. Cavities 227 are formed in the second element E2 of the second CMUT chip C2. Supporters 225 of the second CMUT chip C2 may have a height H2 which is greater than a corresponding height H1 of the supporters 220 of the first CMUT chips C1.

Accordingly, the height H1 of the cavities 222 of the first CMUT chip C1 is formed lower than the second height H2 of the cavities 227 of the second CMUT chip C2. The first height H1 may be in a range from about 10 nm to about 100 nm, and the second height H2 may be approximately 200 nm. The height of the cavities 222 of the first CMUT chips C1 is relatively low, and thus, an electrostatic voltage that is applied to the cavities 222 is reduced, thereby increasing receiving sensitivity.

The first CMUT chips C1 receive low frequency ultrasonic waves, and the second CMUT chips C2 transmit and receive high frequency ultrasonic waves. The first CMUT chips C1 may receive a frequency in a range from about 0.5 to about 4 MHz, and the second CMUT chips C2 may transmit and receive a frequency in a range from about 5 to about 8 MHz. The first CMUT chips C1 receive an acoustic wave which is generated from a diagnosis object by absorbing laser light irradiated from the first and second laser irradiation units 151 and 152. Hereinafter, the acoustic wave may be referred to as a first ultrasonic wave.

A laser light which is emitted from the first and second laser irradiation units 151 and 152 is irradiated onto a texture of a diagnosis object. At this point, a texture in a human body part, such as, for example, blood vessels, has a relatively a high laser light absorption rate, and thus, a thermal expansion and contraction by a laser pulse occurs. Due to the expansion and contraction of the blood vessels, an ultrasonic wave is generated. The first CMUT chips C1 generates an electrical signal by receiving the first ultrasonic wave which is generated from a diagnosis object onto which a laser light is irradiated.

FIG. 2 shows an example of a structure of an ultrasonic transducer chip that is applied to the current exemplary embodiment. The structure of an ultrasonic transducer chip according to the current exemplary embodiment may vary in many forms in addition to the structure depicted in FIG. 2.

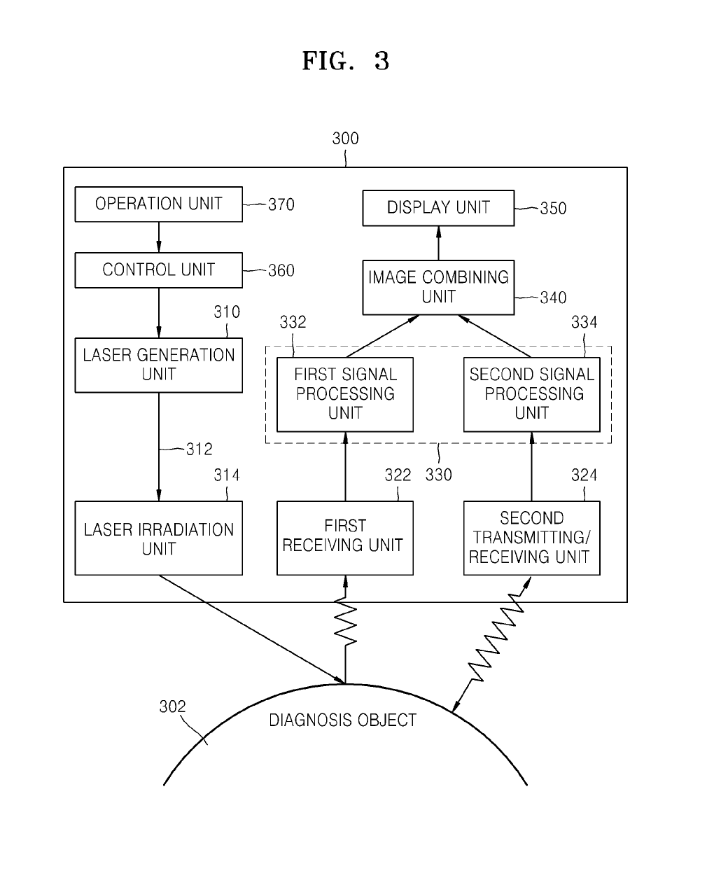

FIG. 3 is a schematic block diagram of a wideband ultrasonic probe 300 for a photoacoustic image and ultrasound image, according to one or more exemplary embodiments.

Referring to FIG. 3, the wideband ultrasonic probe 300 may include a laser generator 310 that irradiates a laser light onto a diagnosis object 302, a first receiving unit (also referred to herein as a “first receiver”) 322 that receives a first ultrasonic wave from the diagnosis object 302, and a second transmitting/receiving (also referred to herein as a “second transmitter/receiver” and/or as a “second transceiver”) unit 324 that transmits a second ultrasonic wave to the diagnosis object 302 and receives the second ultrasonic wave which is an echo signal from the diagnosis object 302.

The laser generator 310 may generate a laser light in a pulse type so that the first ultrasonic wave is generated from the diagnosis object 302. For example, the laser generator 310 may be a solid pulse laser, and a pulse width of the laser light may be in a nano size or a pico size.

A laser irradiation unit (also referred to herein as a “laser irradiator”) 314 receives a laser light from the laser generator 310 via an optical fiber 312, and irradiates a laser light onto the diagnosis object 302. When the laser light is irradiated onto the diagnosis object 302, an acoustic wave, that is, a first ultrasonic wave, is generated as the laser light is absorbed at the texture and/or surface of the diagnosis object 302.

The first receiving unit 322 receives the first ultrasonic wave generated from the diagnosis object 302. The first receiving unit 322 corresponds to the first ultrasonic transducer array 110 of FIG. 1.

The second transmitting/receiving unit 324 transmits a second ultrasonic wave to the diagnosis object 302 and receives the second ultrasonic wave reflected by the diagnosis object 302 by being driven in response to a control signal received from an operation unit (also referred to herein as an “operator”) 370 and a control unit (also referred to herein as a “controller”) 360. The second transmitting/receiving unit 324 corresponds to the second ultrasonic transducer array 120 of FIG. 1.

The electrical signal transformed in the first receiving unit 322 and the second transmitting/receiving unit 324 is an analog signal. The first receiving unit 322 transmits a first electrical signal that is generated by transforming the first ultrasonic wave to a first signal processing unit (also referred to herein as a “first signal processor”) 332, and the second transmitting/receiving unit 324 transmits a second electrical signal generated by transforming the second ultrasonic wave to a second signal processing unit (also referred to herein as a “second signal processor”) 334.

A signal processing unit (also referred to herein as a “signal processor”) 330 may generate an image by processing signals of the first ultrasonic wave and the second ultrasonic wave. For example, the signal processing unit 330 may transform signals provided from the first receiving unit 322 and the second transmitting/receiving unit 324 to digital signals. The signal processing unit 330 generates an image in consideration of locations of each of the elements of the first receiving unit 322 and the second transmitting/receiving unit 324 and the location of the diagnosis object 302. The signal processing unit 330 may perform various signal processing functions (such as, for example, a gain control, and a filtering treatment, etc.) which may be required for forming an image.

The signal processing unit 330 may include the first signal processing unit 332 and the second signal processing unit 334. The first signal processing unit 332 generates a first image by processing a first electrical signal that corresponds to the first ultrasonic wave. The first image may include a photoacoustic image and/or a functional image.

The second signal processing unit 334 generates a second image by processing a second electrical signal that corresponds to the second ultrasonic wave. The second image may include an ultrasound image and/or a morphological image.

An image combining unit (also referred to herein as an “image combiner”) 340 generates a third image by combining the first image with the second image. The combination of the first and second images may be performed based on a specific point of the diagnosis object 302. The combined image may be an image on which the second image is combined the first image that reflects a characteristic of a texture based on a location of the texture and/or a specific surface location. The technique of combining a plurality of images is well known in the art, and thus, the description thereof will be omitted.

A display unit (also referred to herein as a “display” and/or as a “display device”) 350 displays the third image which is generated by the image combining unit 340. The display unit 350 may also display the first image and/or the second image as necessary. Also, the display unit 350 may simultaneously display at least two images from among the first, second, and third images.

The control unit 360 controls constituent elements of the wideband ultrasonic probe 300 based on the user command that is received via the operation unit 370. The control unit 360 may be realized by a microprocessor. The operation unit 370 receives input information from the user. The operation unit 370 may include any one or more of a control panel, a keyboard, and a mouse.

According to one or more exemplary embodiments, after separately manufacturing the photoacoustic image chips and the ultrasound image chips, the photoacoustic image chips and the ultrasound image chips are tiled on a substrate, and laser irradiation units are disposed on both sides of the substrate, thus a wideband ultrasonic probe for a photoacoustic image and an ultrasound image may be readily manufactured.

Because the photoacoustic image chips and the ultrasound image chips are adjacently disposed on a single probe, the co-registration of the photoacoustic image and the ultrasound image may be simplified.

Also, the heights of the cavities of the photoacoustic image chips and the ultrasound image chips may be appropriately designed according to usage, and thus, the sensitivity of the photoacoustic image may be increased by reducing the height of the cavities of the photoacoustic image chips.

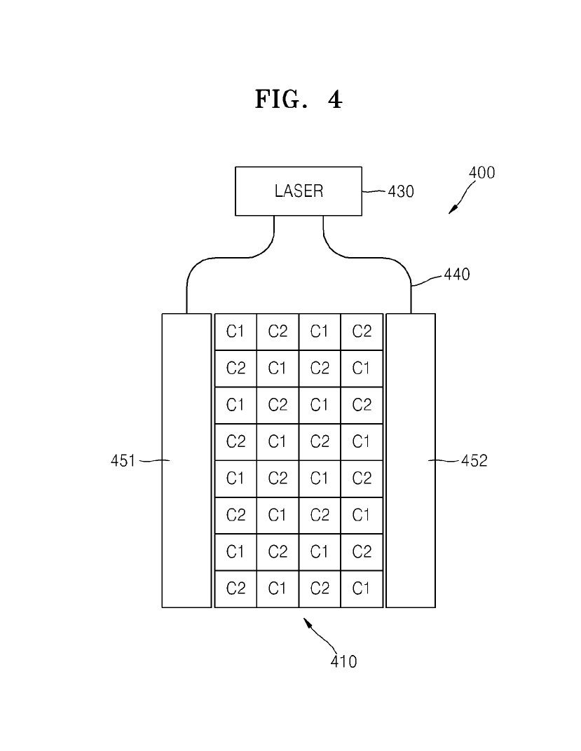

FIG. 4 is a schematic plan view of a structure of a wideband ultrasonic probe 400 for a photoacoustic image and ultrasound image, according to one or more exemplary embodiments.

Referring to FIG. 4, the wideband ultrasonic probe 400 includes first and second laser irradiation units (also referred to herein as “first and second laser irradiators” and/or “first and second laser irradiation components”) 451 and 452 that are disposed on outer regions of an ultrasonic transducer array 410 facing each other. A laser light generated from a single laser generator 430 is divided by optical fibers 440 to the first and second laser irradiation units 451 and 452, and is irradiated onto a diagnosis object. The first and second laser irradiation units 451 and 452 may include optical prisms. The wideband ultrasonic probe 400 may include one of the first and second laser irradiation units 451 and 452. The laser generator 430, the optical fibers 440, and the first and second laser irradiation units 451 and 452 may constitute a laser apparatus.

The laser generator 430 may include a solid pulse laser, such as, for example, an Nd:YAG pulse laser. A pulse width of the laser may be in a nano size or a pico size.

The ultrasonic transducer array 410 includes a plurality of first CMUT chips C1 and a plurality of second CMUT chips C2. Each of the first and second CMUT chips C1 and C2 may include a respective plurality of elements.

The first and second CMUT chips C1 and C2, as shown in FIG. 4, may be alternately disposed. However, the arrangement of the first and second CMUT chips C1 and C2 according to the current exemplary embodiment is not limited thereto, and may be diversely disposed in a determined pattern.

The element of the first CMUT chip C1 may be substantially the same as ore similar to the first element E1 of the first CMUT chip C1 of FIG. 2. Also, the element of the second CMUT chip C2 may be substantially the same as or similar to the second element E2 of the second CMUT chip C2 of FIG. 2.

The structure and operation of the wideband ultrasonic probe 400 may be understood from the exemplary embodiments described above, and thus, the description thereof will be omitted.

In the wideband ultrasonic probe 400, because the first and second CMUT chips C1 and C2 are alternately disposed, location information which relates to the diagnosis object may further be readily shared between the adjacent first and second CMUT chips C1 and C2. Therefore, the co-registration of image information collected from the first and second CMUT chips C1 and C2 may be readily and correctly achieved.

It should be understood that the exemplary embodiments described therein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope of the present inventive concept as defined by the following claims.

What is claimed is:

1. An ultrasonic probe comprising:
a first ultrasonic transducer array and a second ultrasonic transducer array that are disposed on a substrate; and
a laser apparatus that comprises a laser irradiator configured to irradiate a laser light onto a diagnosis object, wherein
the first ultrasonic transducer array is configured to receive a first ultrasonic wave which is generated from the diagnosis object on which the laser light is irradiated, and the second ultrasonic transducer array is configured to transmit a high frequency bandwidth ultrasonic wave toward the diagnosis object and to receive a second ultrasonic wave that is reflected by the diagnosis object.

2. The ultrasonic probe of claim 1, wherein the first ultrasonic transducer array comprises a plurality of first ultrasonic transducer chips, and the second ultrasonic transducer array comprises a plurality of second ultrasonic transducer chips, wherein each of the plurality of first ultrasonic transducer chips and each of the plurality of second ultrasonic transducer chips is a capacitive micromachined ultrasonic transducer (CMUT) chip.

3. The ultrasonic probe of claim 2, wherein each of the plurality of first ultrasonic transducer chips is disposed on a first region of the substrate and each of the plurality of second ultrasonic transducer chips is disposed on a second region of the substrate, and the second region is adjacent to the first region.

4. The ultrasonic probe of claim 2, wherein the plurality of first ultrasonic transducer chips and the plurality of second ultrasonic transducer chips are alternately disposed.

5. The ultrasonic probe of claim 2, wherein the first ultrasonic transducer array is further configured to receive a frequency bandwidth in a range from about 0.5 MHz to about 4 MHz.

6. The ultrasonic probe of claim 2, wherein the second ultrasonic transducer array is further configured to receive a frequency bandwidth in a range from about 5 MHz to about 18 MHz.

7. The ultrasonic probe of claim 2, wherein a cavity between an upper electrode and a lower electrode of the first ultrasonic transducer chip has a first height that is smaller than a second height of a cavity between an upper electrode and a lower electrode of the second ultrasonic transducer chip.

8. The ultrasonic probe of claim 7, wherein the first height is in a range from about 10 nm to about 100 nm.

9. The ultrasonic probe of claim 1, wherein the laser apparatus includes a pulse laser.

10. The ultrasonic probe of claim 9, wherein the pulse laser has a pulse width which is in a range of between about 1 picosecond and 1000 nanoseconds.

11. The ultrasonic probe of claim 1, wherein the laser irradiator comprises a first laser irradiation component which is disposed at an outer region of the first ultrasonic transducer array and a second laser irradiation component which is disposed at an outer region of the second ultrasonic transducer array,
wherein the first laser irradiation component is disposed to face the second laser irradiation component such that the first ultrasonic transducer array and the second ultrasonic transducer array are disposed between the first laser irradiation component and the second laser irradiation component.

12. The ultrasonic probe of claim 1, further comprising:
a first signal processor configured to generate a first image by receiving, from the first ultrasonic transducer array, an electrical signal which corresponds to the first ultrasonic wave;
a second signal processor configured to generate a second image by receiving, from the second ultrasonic transducer array, an electrical signal which corresponds to the second ultrasonic wave; and
an image combiner configured to generate a third image by combining the first image with the second image.

13. The ultrasonic probe of claim 12, further comprising a display device configured to display at least one from among the first image, the second image, and the third image.
